(19) United States
(12) Patent Application Publication
(10) Pub. No.: US 2021/0393384 A1
(43) Pub. Date: Dec. 23, 2021

(54) DEVICE FOR LOCATING AN APEX FOR MEASURING THE CONCENTRATION OF AN IRRIGATION SOLUTION IN THE APICAL ZONE OF A DENTAL ROOT CANAL

(71) Applicant: THIERRY MAILLARD, LE HAILLAN (FR)

(72) Inventor: THIERRY MAILLARD, LE HAILLAN (FR)

(21) Appl. No.: 17/354,778

(22) Filed: Jun. 22, 2021

(30) Foreign Application Priority Data
Jun. 22, 2020 (FR) 20 06486

(51) Int. Cl. A61C 19/04

(52) U.S. Cl. CPC A61C 19/042

(57) ABSTRACT

A device to electronically locate an apex in a root canal of a tooth. The device is configured to be electrically connected to an endodontic instrument engaged in the root canal and to a labial electrode. The device includes a screen to display the progression of the instrument in the root canal relative to a critical apical point of reference, such as the cemento-dentinal junction. The device is configured to measure, in the vicinity of the point of reference, by conductimetry, a concentration of an electrolyte irrigation solution injected into the root canal.

### RELATED APPLICATION

This application claims priority from French Patent Application No. 20 06486 filed Jun. 22, 2020, which is incorporated herein by reference in its entirety.

### TECHNICAL FIELD

The present invention belongs to the general field of endodontics, in particular devices for locating an apex, commonly called (electronic) apex locators, and relates more particularly to an apex locator making it possible to measure the concentration of the irrigation solution in the root canal before the cemento-dentinal junction.

Such an apex locator is for example used during the step of canal preparation of the endodontic treatment.

### RELATED ART

It is suitable beforehand to briefly recall the anatomy of a dental root at the apical zone. The canal system and more particularly the apical structure can for example be described according to Kuttler's apical concept which gives a rather representative model of it diagramed in FIG. 1.

FIG. 1 shows, as a partial longitudinal cross-section, a dental root DR passed through at its center by a root canal RC which terminates with an orifice AF, called apical foramen, at the end of said root for the passage of blood vessels and nerve fascicles. The apical foramen AF generally has a bottleneck shape due to a shrinking then an enlarging of the root canal RC which define a zone of minimum diameter called apical constriction AC. The apical constriction AC constitutes the narrowest passage of the neurovascular bundle irrigating the pulp. Generally, the root canal RC has a converging then diverging shape between the coronary part and the periapex, which amounts to two cones opposed by their vertices at the apical constriction AC. More precisely, the root canal RC defines a dentinal cone and a shorter cemental cone, the interface between the two is called cemento-dentinal junction CDJ. As shown in the detail A of FIG. 1, the apical constriction AC and the cemento-dentinal junction CDJ are not necessarily confounded, but it happens that they are. Specialized literature shows that the AC regularly coincides with the CDJ or is in the vicinity of the latter.

During an endodontic treatment, the practitioner (dental surgeon) seeks to remove all the materials, debris and organic fluids that fill up the root canal to the bottom, i.e., to the apical foramen, in order to prevent a dental abscess from appearing again in said canal. However, their objective is especially, as much as possible, to not go beyond the apical terminus APX, or apex, on the one hand, to not cause pain to the patient, and on the other hand, to not dig a cavity under the root, beyond the apex, which could give rise to the appearance of an abscess.

It is therefore primordial for the practitioner to very precisely locate the apical foramen and the apex, in particular during operations of cleaning and shaping the root canal (canal preparation), in order to prevent crossing the apical foramen. This reverts to determining “the working length” which designates the exact length between a coronary point of reference and an apical limit fixed and chosen beforehand, conventionally located at the apical constriction. This working length can be determined using a radiography (which gives the less precise radiographic apex RA) and/or an electronic apex locator (which gives the more precise anatomical apex AA).

Locating the apex also makes it possible to correctly conduct the irrigation, which is a key step in canal preparation.

The objective of the irrigation is the removal of various microorganisms, lubrification of the endodontic instruments inserted into the canal and dissolution of the organic debris and minerals. It consists of abundantly injecting into the canal an irrigation solution such as sodium hypochlorite (NaOCl).

Sodium hypochlorite has an antimicrobial action and dissolution of tissues of which the effectiveness increases with the concentration, but has a risk of toxicity, in particular of cytotoxicity, at high concentrations.

“Medical” concentrations of NaOCl ranging from 0.5% to 5.25% are widely used in clinical practice. Although less concentrated solutions have shown a certain antimicrobial effectiveness, the aforementioned concentrations have a faster and more substantial bactericidal effect, but in general must not be exceeded.

At an excessively high concentration, sodium hypochlorite has an extremely toxic effect on vital tissues, it is a corrosive liquid. Propelled in a substantial quantity into the periapex, it can cause effects such as hemolysis, an ulceration of the skin, necrosis, even a weakening of the facial nerves of the region concerned. In addition, sodium hypochlorite has an effect on the dentinal hardness. Prolonged exposure at a high concentration can decrease the elasticity modulus (in flexure) of the dentin.

It is therefore capital to know with precision the concentration of the irrigation solution, here NaOCl, in the canal, especially in the apical zone, to ensure effective treatment and limit any risk of toxicity.

No apex locator or similar, as far as the applicant knows, makes it possible to determine this concentration in the apical zone, in particular at the cemento-dentinal junction.

### OBJECT AND SUMMARY OF THE INVENTION

The present invention aims to respond to the needs of practitioners to know in a practical and reliable manner the concentration of the irrigation solution in the root canal during an endodontic treatment.

To that end, the object of the present invention is an electronic device for locating an apex in a root canal of a tooth, able to be electrically connected to an endodontic instrument engaged in the canal and to a labial electrode, and including a screen to display a progression of the instrument in said canal relatively to a critical apical point of reference such as the cemento-dentinal junction. This device is remarkable in that it is furthermore able to measure, in the vicinity of said point of reference, by conductimetry, a concentration of an electrolyte irrigation solution injected into the root canal.

According to a particularly advantageous aspect of the invention, any measurement of the concentration of the irrigation solution is taken in the last millimeter before the cemento-dentinal junction in the direction of engagement of the endodontic instrument in the root canal.

According to an embodiment, a measured level of concentration, relatively to a range of medical concentrations, is displayed on the screen of the device via a visual indicator.

For example, the visual indicator is a bar graph.

Furthermore, the device includes connection cords via which it can be electrically connected to the endodontic instrument, to the labial electrode and the like.

Advantageously, processing and calculation means of the device are adapted for a conductimetric calculation at least for one irrigation solution of sodium hypochlorite NaOCl, and make it possible to display a measured level of concentration on a scale that comprises at least one value substantially equal to: 0.5%, 2.5% or 5.25%, with these concentrations being the most recommended and used in endodontics, in particular during canal preparations.

According to an embodiment, the screen displays validation information of the “OK” type as long as the measured concentration of the irrigation solution is substantially equal to an injection concentration of said solution.

The object of the invention is also a method for measuring the concentration of an irrigation solution in a root canal of a tooth implementing a device such as shown.

Advantageously, the method comprises steps of memorizing concentration information in order to facilitate the work of the practitioner because they can read the value of the concentration of the irrigation solution even when the instrument is out of the canal of the patient.

As the fundamental concepts of the invention have just been disclosed in their most elementary form, other details and characteristics shall appear more clearly when reading the following description and with respect to the accompanying drawings, giving by way of a non-limiting example an embodiment of a device in accordance with the principles of the invention.

### BRIEF DESCRIPTION OF THE DRAWINGS

The figures are given solely for the purposes of illustration for the intelligence of the invention and do not limit the scope of the latter. The different elements are shown diagrammatically and are not necessarily on the same scale.

It is thus shown as:

FIG. 1 is a longitudinal cross-section of a dental root at the apical zone according to Kuttler's apical concept;

FIG. 2 is an example of using a device for locating an apex according to the invention on a tooth to measure the concentration of the irrigation solution;

FIG. 3 is the principle of conductimetric measurement of the concentration of the irrigation solution in the vicinity of the cemento-dentinal junction;

FIG. 4 is a simplified electrical assembly that makes it possible to measure the concentration by conductimetry according to the invention;

FIG. 5a is an example of a graphical interface for monitoring the progression of the instrument in the canal and the concentration of the irrigation solution, according to the state of progress of the canal preparation, here the instrument is outside the measurement zone in the vicinity of the critical apical point of reference;

FIG. 5b is the graphical interface when the instrument crosses the last millimeter before the cemento-dentinal junction; and

FIG. 5c is the graphical interface when the instrument is at the apex.

### DETAILED DESCRIPTION OF EMBODIMENTS

It is suitable to note that certain devices and methods well known to those skilled in the art are here described to prevent any insufficiency or ambiguity in the comprehension of the present invention.

In the embodiment described hereinafter, reference is made to an improved device for locating an apex to measure the concentration of the irrigation solution in the root canal of a tooth, mainly intended for measuring said concentration in the immediate vicinity of the cemento-dentinal junction during a canal preparation. This example, which is not limiting, is given for better comprehension of the invention and does not exclude another use of the device. For example, the device can be adapted to measure the concentration of an irrigation solution or of a physiological liquid in another anatomical region of which the treatment is similar to the endodontic treatment, at least regarding the injection of an ionic solution into a canal terminating with a tip (apex).

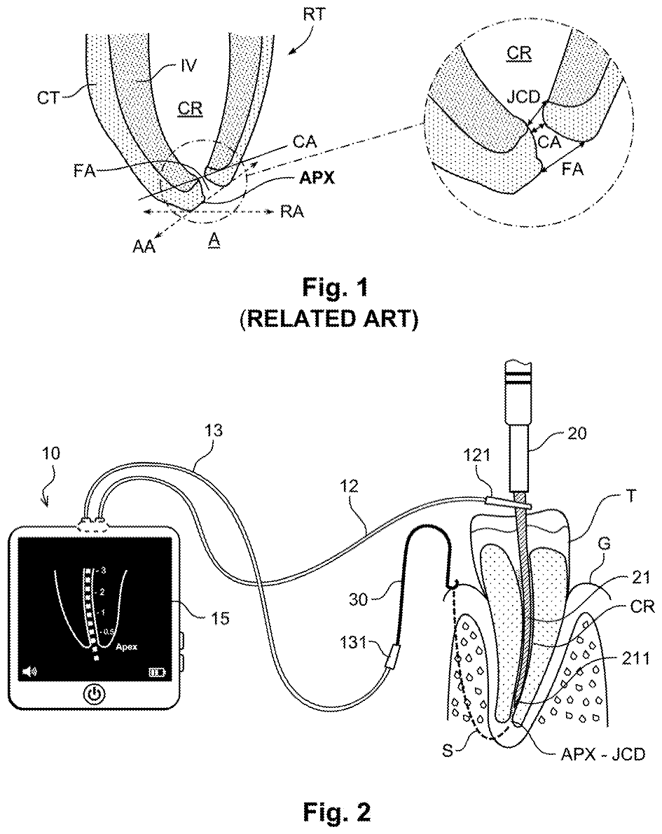

In the rest of the description, the cemento-dentinal junction is designated by its acronym CDJ; the term “irrigant” designates an irrigation solution; and the term “apex locator” designates by extension an electronic apex locator. Any reference to an anatomical part of the dental root at the apical zone will be done in reference to FIG. 1 already described.

FIG. 2 shows an apex locator 10 conventionally connected on a tooth T specimen, or on a dental canal model, via an endodontic instrument 20, such as a file, and a labial hook 30, both having electrically conductive parts connected to the apex locator by connection cords 12 and 13 in such a way as to form two measuring electrodes.

According to a known principle, the apex locator 10 makes it possible to locate the apex in the root canal RC of the tooth T using as a basis an indirect measurement of the resistance, or more generally of the impedance, by application of Ohm's Law between the terminals defined by the two electrodes.

As a general rule, apex locators make use of the observation according to which the root canal has a fixed electrical impedance at the apical constriction, and operate with an alternating electric current in a closed circuit thanks to two electrodes delimiting a conductive anatomical unit between the canal and the lip of the patient. As the electrical voltage is known, imposed by the generator of the apex locator, the latter calculates the intensity of the current flowing, the latter varies according to the impedance of the anatomical unit. Thus, during the crossing of the apical zone by the instrument used, the value of the impedance changes abruptly to reach a value that is substantially constant from one individual to another, and the locator detects the intensity of the corresponding current.

The apex locator 10 according to the present invention makes use of this principle and the link between electrical impedance and conductance to determine the concentration of an irrigation solution in the canal by conductimetry, as explained hereinafter.

The apex locator 10, according to the example shown, comprises a display screen 15 whereon different pieces of information can be read to allow for real-time monitoring of the penetration of the endodontic instrument 20 in the root canal RC, and this, until the apex is detected.

The connection cords 12 and 13 of the apex locator 10 are provided at their free end with a clamp 121 or any other means of connection 131 to be attached to the electrically conductive parts of the endodontic instrument 20 and of the labial hook 30.

The endodontic instrument 20, according to the example shown, is a canal preparation file including a metal rod 21 ending with a tip 211. Such a file is more preferably flexible in order to adapt to root canals with different curvatures. The clamp 121 of the connection cord 12 is fixed on the metal rod 21 of the file 20.

The labial hook 30 is however shaped to ensure a contact with the lip of the patient and/or their gingiva G.

Therefore, the endodontic instrument 20 and the labial hook 30 electrically delimit an anatomical unit passed through by the electric current flowing, shown as a broken line S in FIG. 2.

According to a fundamental aspect of the present invention, the apex locator 10, thanks to this conventional mounting, furthermore makes it possible to measure the concentration of the irrigation solution in the canal RC, at the tip 211 of the instrument 20, via a conductimetry technique.

Indeed, the irrigation solution is an electrolyte solution wherein bathe charge carriers (ions) in aqueous form produced by the chemical reaction of the irrigation solution in the canal medium.

The case of sodium hypochlorite NaOCl is considered here which currently remains the most commonly used irrigant, even inevitable, in endodontics.

The antiseptic and dissolving effect of NaOCl is obtained by the following successive reactions:

NaOCl+H₂O↔NaOH+HOCl↔Na⁺+OH⁻+H⁺+OCl⁻.

The first is a dissolution reaction of the sodium hypochlorite which produces hypochlorous acid HOCl. The latter, in a basic medium, is dissociated during the second reaction and produces hypochlorite ions OCl⁻ known for their antibacterial effect. Indeed, the hypochlorite ion is a strong oxidant that inhibits bacterial enzymes and leads to an irreversible oxidation of the sulfhydryl groups (SH) which form most of the so-called enzyme membranes, thus causing the destruction thereof.

Of course, the ions formed after the second reaction hereinabove are aqueous.

Due to the presence of electrically-charged chemical species, the conductivity a of the irrigation solution, in the case of NaOCl, is written:

σ=λ_{Na}_{⁺}[Na⁺]+λ_{OH}_{^{−}}[OH⁻]+λ_{H}_{⁺}[H⁺]+λ_{OCl}_{^{−}}[OCl⁻]

with λ_{X} the ionic molar conductivity of the ion X, and [X] the concentration thereof in the solution.

By noting as C the concentration of the solution in chemical equilibrium, and in light of the charge coefficients of the ions at play, it is possible to write:

σ=CΣᵢλᵢ.

However, it is known that the conductivity is proportional to the conductance G which is none other than the inverse of the resistance R according to the formulas:

$$σ = k G ; G = \frac{1}{R} .$$

The coefficient k depends on the geometry of the measuring cell.

By noting as Λ the sum of the ionic molar conductivities, it can be established that:

$$C = \frac{σ}{Λ} = \frac{k}{Λ R} = \frac{{k}^{′}}{R} .$$

The concentration C is therefore inversely proportional to the resistance R, the proportionality coefficient k′ is a constant that essentially depends on the nature of the ions in the irrigation solution and on the geometry of the measuring cell.

The simplified formulation hereinabove is given to show the link between the resistance (or the impedance) of the anatomical unit passed through by the electric current and the concentration of the irrigation solution, a link that makes it possible to use the basic principle of the apex locator to determine said concentration by conductimetry.

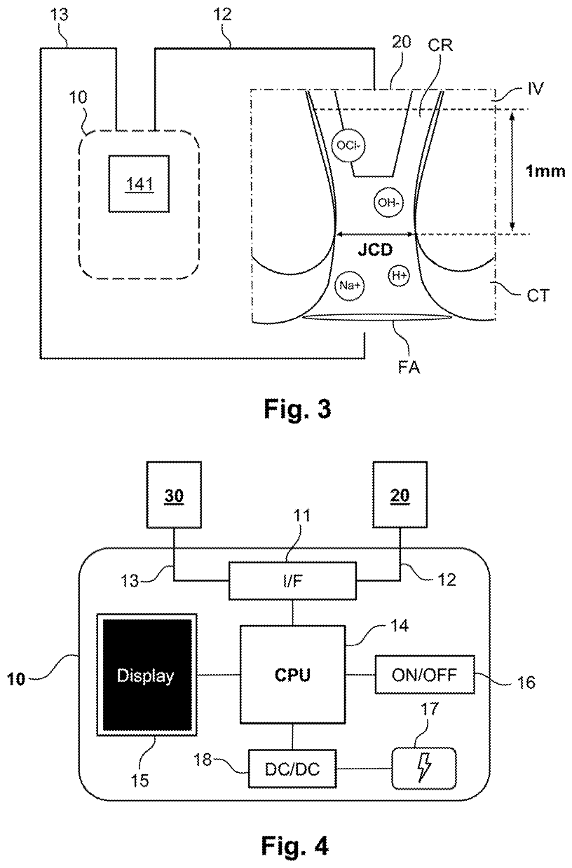

FIG. 3 diagrammatical shows the measurement principle of the concentration of the irrigant in the apical zone of a dental root of which the anatomical structure has been simplified for better comprehension.

During the irrigation, the NaOCl solution fills the root canal RC up to the apical foramen AF and penetrates even into the least crevices of the canal thanks to its low surface tension.

The objective of the present invention is to allow the practitioner to know the concentration of the irrigant in the vicinity of the CDJ, for example in the last millimeter before the CDJ.

As a general rule, canal cleanings are carried out with concentrations of NaOCl between 0.5%, 2.5% and 5.25%. Consequently, the apex locator 10 can be calibrated to indicate the level of concentration measured in this range of so-called “medical” concentrations.

Among its means of calculating and processing, the apex locator 10 can include a specific electronic module 141 to manage the conductimetric calculations independently of the basic function of said locator, namely the detection of the apex. Such a module could be in the form of an electronic board that can be easily integrated into known models of apex locators.

Preferably, the conductimetric calculations are directly carried out by a central calculator of the locator 10 as explained hereinafter in reference to FIG. 4.

As long as the operation of the electronic apex locators is based on the variation of an electric current according to the specific impedance of the canal, and the latter depends directly on the concentration of a possible electrolyte filling the canal, such as the irrigant, the present invention is adapted to the different generations and technologies of apex locators.

In particular and according to the embodiment of FIG. 4, the apex locator 10 includes a connection interface 11 with the electrodes 20 and 30 through the connection cords 12 and 13, a calculation unit 14 of the microprocessor type connected to the different services in particular the display module 15, an on/off button 16, an electrical power supply battery 17 as well as an alternating current regulator 18.

The microprocessor 14 uses both the apex locating calculations and the conductimetric calculations to measure the concentration of the irrigation solution. Of course, the apex locator has memory means required for storing the information generated.

It is important to recall that the quality of a canal cleaning mainly depends on the concentration of the irrigant (in the aforementioned medical range) and on the time the irrigant is present in the canal. Thus, the practitioner needs to monitor the concentration of the irrigant in the canal when they carry out a canal cleaning via irrigation. All the more so as knowledge of this concentration in the vicinity of the CDJ is crucial for ensuring the effectiveness of the treatment in this zone prone to complications and the appearance of abscesses.

Therefore, the apex locator 10 according to the present invention allows for fast and intuitive access to the information on the concentration of the irrigant in the apical zone of the canal.

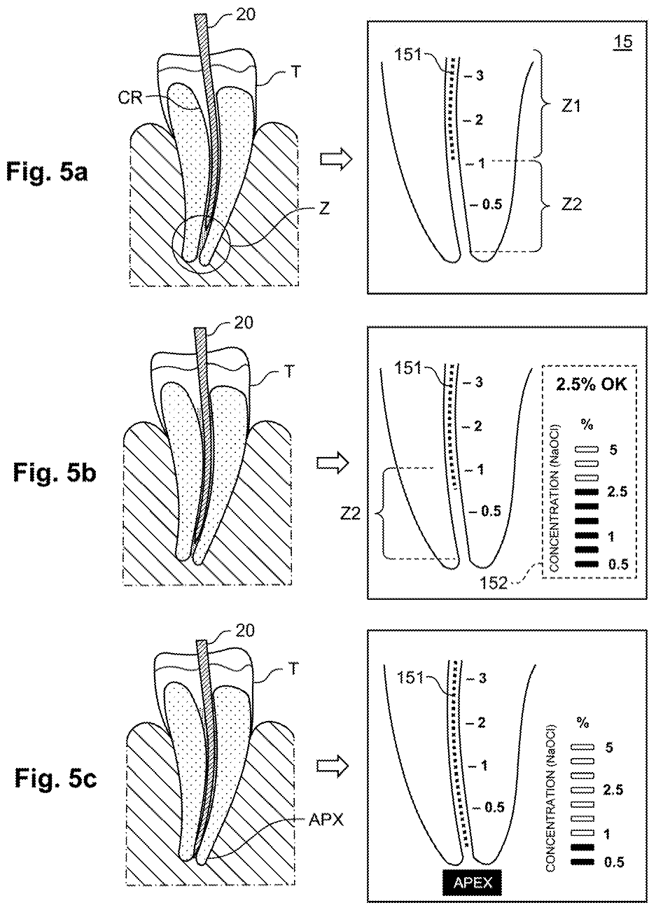

FIGS. 5a to 5c give a possible display example (graphical interface) as the endodontic instrument 20 progresses in the canal RC of the tooth T. This display makes it possible to visualize, roughly, a partial longitudinal section of the dental root, at the apical zone Z, whereon a progression line 151 as a broken line indicates the progression of the instrument 20 inside the root canal RC. According to the example shown, the apical zone displayed corresponds approximately to the last three millimeters of the root, more precisely to the last three millimeters before the CDJ. The scale factor used is preferably dilated over the millimeter for better monitoring in this critical zone of the endodontic treatment.

In FIG. 5a, the instrument 20 begins to engage in the apical zone Z of the root canal RC. The display obtained on the screen 15 of the apex locator shows that the progression line 151 has not yet entered into the last millimeter before the CDJ which corresponds to the critical zone wherein the concentration of the irrigant has to be known. Indeed, it is possible to isolate two zones on the cross-section of the root displayed: a zone Z1 wherein the measurement of the concentration is not required and therefore not carried out, and a zone Z2 wherein this measurement is required in order to not leave any doubt for the practitioner during the canal preparation. The zone Z1 is located upstream from the last millimeter, while the zone 2 corresponds to this last millimeter before the CDJ. In addition, during an injection of the irrigant at a given concentration, the irrigant fills the zone Z1 without difficulty and its concentration remains generally constant, which makes measuring said concentration unnecessary in this zone. On the other hand, the filling of the zone Z2 in the vicinity of the CDJ (and therefore often in the vicinity of the apical constriction) by the irrigation solution remains problematic and uncertain due to the narrowness of the canal in this zone. That is why it is of interest to measure the concentration in this critical zone.

In the example of FIGS. 5a to 5c, an injection of a solution of sodium hypochlorite NaOCl at a concentration of 2.5% is considered.

FIG. 5b shows that the instrument 20 has crossed the apical zone considered in such a way that the display obtained indicates the presence of the progression line 151 in the concentration measurement zone Z2. The level of concentration measured is therefore indicated on the screen of the apex locator via a visual indicator such as a bar graph 152. For example, the display of the level of concentration can appear automatically and abruptly on the screen as soon as the instrument enters into measurement zone Z2, in order to visually alert the practitioner. In the example shown, the screen indicates a level of concentration of 2.5%, in accordance with the injection concentration. A monitoring message such as “OK” can also be displayed when the concentration is at the level required so that the practitioner user is not disturbed by a complicated reading of the information. Thus, the practitioner monopolizes a major portion of their vigilance in the manipulating of the endodontic instruments inside the canal.

Furthermore, memorizing the concentration value would allow for easy reading even after having removed the canal instrument. Resetting the information would occur when introducing the instrument in the canal again or during the automatic shutdown of the locator.

In FIG. 5c, the instrument 20 has reached the apex or, at least, is very close to it, within permissible tolerances, and the screen displays a progression line 151 that extends to “zero” of the millimetric point of reference. The apex locator can report the proximity of the instrument 20 with the apex via a visual and/or audible notification. According to the example shown, the concentration of the irrigant is less than 1% and therefore remains below the required value. In this case, the practitioner has to renew the irrigation of the canal by adding a certain quantity of solution in order to increase the concentration of the irrigant in this zone. Moreover, the screen does not display any negative message when the concentration is not sufficient in order to prevent any confusion and make the graphical interface as intuitive as possible.

However, simplified example of the graphical interface shown hereinabove is not limiting, said interface can be modified according to the recommendations of the users to which the apex locator is addressed.

Finally, it is clear from the present description that certain elements of the apex locator can be modified, suppressed or replaced without however leaving the scope of the invention, defined in the claims.

1-7. (canceled)

8. A device to electronically locate an apex in a root canal of a tooth, the device being configured to electrically connect to an endodontic instrument engaged in the root canal and to a labial electrode, the device comprising a screen to display a progression of the endodontic instrument in the root canal relative to a critical apical point of reference, and the device configured to measure, in the vicinity of the critical apical point of reference, by conductimetry, a concentration of an electrolyte irrigation solution injected into the root canal.

9. The device of claim 8, wherein the critical apical point of reference is a cemento-dentinal junction.

10. The device of claim 9, wherein a measurement of the concentration of the electrolyte irrigation solution is taken in a last millimeter before the cemento-dentinal junction in a direction of engagement of the endodontic instrument in the root canal.

11. The device of claim 8, wherein a measured level of concentration, relative to a range of medical concentrations, is displayed on the screen via a visual indicator.

12. The device of claim 11, wherein the visual indicator is a bar graph.

13. The device of claim 8, further comprising connection cords to electrically connect the device to the endodontic instrument and to the labial electrode.

14. The device of claim 8, further comprising a microprocessor configured to perform a conductimetric calculation at least for one irrigation solution of sodium hypochlorite, and display a measured level of concentration on a scale that comprises at least one value substantially equal to: 0.5%, 2.5% or 5.25%.

15. The device of claim 8, wherein the screen displays validation information in response to a determination that a measured concentration of the irrigation solution is substantially equal to the concentration of the electrolyte irrigation solution injected into the root canal.
